(19) United States
(12) Patent Application Publication
(10) Pub. No.: US 2025/0072832 A1
(43) Pub. Date: Mar. 6, 2025

(54) SYSTEM AND METHOD FOR IMPROVING HYPERSOMNIA DIAGNOSTIC ACCURACY OF SLEEP ONSET DETECTION

(71) Applicant: HAPPY HEALTH, INC., Austin, TX (US)

(72) Inventor: Conrad W. Rosenbrock

(21) Appl. No.: 18/822,144

(22) Filed: Aug. 31, 2024

Related U.S. Application Data
(60) Provisional application No. 63/579,994, filed on Sep. 1, 2023.

(51) Int. Cl. A61B 5/00

(52) U.S. Cl. CPC A61B 5/6826

(57) ABSTRACT

A computer-implemented method is disclosed. The method includes (i) receiving a plurality of sensor streams, each sensor stream is received from a sensor of a plurality of sensors of a wearable electronic device; (ii) determining a sampling rate of each sensor stream; (iii) identifying a count of gaps and a length of each gap for each sensor stream; (iv) identifying a subset of a circular buffer for each sensor of the plurality of sensors that fits within a slice of time of interest characterized by a start timestamp and a stop timestamp; (v) determining consensus between the plurality of sensor streams; (vi) adding the plurality of sensor streams to the circular buffer; and (vii) determining whether a valid window of data exists for the plurality of sensor streams added to the circular buffer for synchronizing two or more sensor streams for a real time application.

### CROSS-REFERENCE TO RELATED APPLICATION

This application is a nonprovisional and claims the benefit under 35 U.S.C. § 119(e) of U.S. Provisional Patent Application No. 63/579,994, filed Sep. 1, 2023, the contents of which are incorporated herein by reference as if fully disclosed herein.

### TECHNICAL FIELD

Embodiments described herein generally relate to a system and a method synchronizing real time raw sensor data from multiple sensors included in a wearable electronic device.

### BACKGROUND

Wearable electronic devices such as smart watches, a wearable ring, fitness trackers, glucose trackers, etc., include sensors for continuous monitoring of physiological and/or biological signals of a user when a wearable electronic device is worn by the user. The sensors included in the wearable electronic device monitor health-related signals such as step count, heart rate, blood glucose level, blood pressure, respiratory rate, galvanic skin conductance (or electrodermal activity (EDA)), sleep patterns, skin temperature, and many others. Sensor data, based upon the monitored signals, helps the user to manage health status and empower the user in self-management of chronic disease conditions for diabetes, obesity, hypersomnia, and cardiovascular disease by incorporating certain lifestyle changes. When multiple sensors included in the wearable electronic are producing sensor data, the actual sample rates differ depending on a variety of factors including the sensor configuration, temporary hardware failures, and/or communication protocol failures, etc. In certain applications, sensor data from multiple sensors are required and if the sensor data is not synchronized with respect to time and other aspects, output accuracy degradation of the applications can occur.

This section is intended to introduce the reader to various aspects of art that may be related to various aspects of the present disclosure described or claimed below. This description is believed to be helpful in providing the reader with background information to facilitate a better understanding of the various aspects of the present disclosure. Accordingly, it should be understood that these statements are to be read in this light and not as admissions of prior art.

### SUMMARY

In one aspect, a computer-implemented method is disclosed. The computer-implemented method includes . . . .

In another aspect, a wearable electronic device is disclosed. The wearable electronic device includes a body, at least one memory storing instructions, at least one processor communicatively coupled with the at least one memory, and a plurality of sensors disposed at or within the body. The plurality of sensors includes one or more of a thermistor, an EDA sensor, a PPG sensor, an accelerometer, and/or an inertial measurement unit (IMU) sensor. The at least one processor is configured to execute the instructions to perform operations comprising: . . . .

In yet another aspect, a wearable electronic device is disclosed. The wearable electronic device includes an annular body, at least one memory storing instructions, at least one processor communicatively coupled with the at least one memory, and a plurality of sensors disposed at or within the annular body. The plurality of sensors includes one or more of a thermistor, an EDA sensor, a PPG sensor, an accelerometer, and/or an inertial measurement unit (IMU) sensor. The at least one processor is configured to execute the instructions to perform operations comprising: . . . .

Various refinements exist of the features noted in relation to the above-mentioned aspects. Further features may also be incorporated in the above-mentioned aspects as well. These refinements and additional features may exist individually or in any combination. For instance, various features discussed below in relation to any of the illustrated examples may be incorporated into any of the above-described aspects, alone or in any combination.

### BRIEF DESCRIPTION OF DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1A illustrates an example wearable electronic device;

FIG. 1B illustrates an example block diagram of the wearable electronic device shown in FIG. 1A; and

FIG. 2 is a flow-chart of method operations for . . . using the wearable electronic device shown in FIG. 1A or FIG. 1B.

Corresponding reference characters indicate corresponding parts throughout the several views of the drawings. Although specific features of various examples may be shown in some drawings and not in others, this is for convenience only. Any feature of any drawing may be referenced or claimed in combination with any feature of any other drawing.

Some structural or method features may be shown in specific arrangements and/or orderings in the drawings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments, and, in some embodiments, it may not be included or may be combined with other features.

### DETAILED DESCRIPTION

Reference will now be made in detail to representative embodiments/aspects illustrated in the accompanying drawings. It should be understood that the following description is not intended to limit the embodiments to one preferred embodiment. On the contrary, it is intended to cover alternatives, modifications, and equivalents as can be included within the spirit and scope of the described embodiments as defined by the appended claims.

Various embodiments are described herein for synchronizing real time raw sensor data (or real time raw sensor readings) from multiple sensors so that the raw sensor readings are in consensus with each other with respect to time, window size, minimum window size, and/or minimum number of samples. The logical conditions presented in the disclosure are primarily intended to synchronize sensor streams (or raw sensor readings) at the hardware level, the same logical conditions and method for synchronizing raw sensor data may be applied at multiple stages in any wearable technology that relies on multiple sensors streaming data concurrently or in near real time.

When multiple sensors in a wearable electronic device are producing data, the actual sample rates of sensor data of different sensors can differ depending on a variety of factors including, but not limited to, sensor configurations, temporary hardware failures, and/or communication protocol failures, etc. When performing real time analysis of signals, the data arriving from multiple sensors is combined in a way that represents real life. For example, if the EDA sensor data (or EDA sensor stream) has a gap in its reported samples due to an error but the accelerometer sensor data (or accelerometer sensor stream) does not have a gap, EDA sensor stream and accelerometer sensor stream cannot be related only to the number of samples (which assumes an ideal sample rate and no real-life hardware failures). Combining multiple sensor streams in real time is a difficult problem for reasons such as, (i) individual sensors have varying sample rates and error rates; (ii) gaps in the data that need to be handled correctly; (iii) consensus that needs to be reached between the sensor streams to ensure that a large enough window in time (or number of samples) has been reached to warrant reporting anything; (iv) memory constraints requiring circular buffers; (v) the time of samples within a given frame being different from the time at which the frame arrives; (vi) arrival of sensor frames with data before and after the gap at the same time when a gap occurs in a sensor stream; (vii) reporting time differences between sensor frames of different sensors causing one group of sensors to report sensor frames earlier or quickly than another group of sensors (e.g., a sensor reporting a single value every 5 seconds compared to an accelerometer reporting 3 values at 125 Hz), thereby introducing lead/lag in the sensor group consensus. Additionally, or alternatively, presence of hardware failures may add to the difficulties of combining multiple sensor streams.

If a leading sensor hits a gap at the same time as a lagging sensor, a race condition may arise where each sensor waits for the other sensor to get out of the gap. However, it may happen neither of the leading or lagging sensor will be able to get out of the gap. Further, higher group-level statistics and checks are required to ensure that the buffering does not get stuck in a loop for indefinite time period. Accordingly, various embodiments in the present disclosure provide a robust real time buffering system that correctly handles gaps and failures at differing sampling rates with consensus mechanisms in place in different conditions causing gaps in sensor streams.

In some embodiments, the real time buffering system may support the following options as merely merging the time series data from multiple sensor streams is not enough.

Window: Window refers a maximum length (in seconds) of a returned window.

Stride: Stride refers to time by which the entire window slides forward in time after a window is found and before trying to find another window.

Minimum Window: Minimum window refers to a minimum length (in seconds) of a returned window. As soon as enough data is available to satisfy this option, the buffer will begin to return aligned sensor data. The number of seconds of data returned will increase as new frames are added until Window (the maximum length (in seconds)) is reached. After that point in time, the data will always have a length of Window seconds until a gap in the data is hit. At that point, the window will begin to shrink again until more data appears after the gap and the window begins to grow again, and/or the gap becomes so large that there is not even Minimum Window seconds of data left, in which case no data is available to return.

Minimum Samples: Minimum Samples refers to a minimum number of samples of data that must exist for the window to be useful. If any of the sensors has fewer than the minimum samples, then the window is rejected.

Align Tolerance: Align Tolerance refers to how close the sensors' window start and/or stop times have to be before one is considered leading or lagging. Generally, a value of align tolerance is around 2 seconds. Accordingly, if a first sensor's start time for a window is more than 2 seconds ahead of a start time of a second sensor, then the first sensor is leading over the second sensor.

Minimum New Window: Minimum new window refers to a minimum number of seconds of new data that must be present in each sensor's window for the data to be useful. If there is a full window of data, which then hits a gap, the returned window shrinks until less than minimum window seconds of data. However, as the window shrinks, the retuned data is seen before (because of the gap). Thus, the amount of new data is zero. For algorithms requiring new data in every window, this is unacceptable.

In an example embodiment, buffering may be optionally controlled so that data windows are only returned when new data is present. Accordingly, new classes of algorithms may be supported when data from multiple sensors is present.

Additionally, or alternatively, each sensor in a group of sensors may have following additional options. The group of sensors includes two or more sensors whose sensor streams are required to be synchronized, as described herein.

Required: when true, if a sensor doesn't have data within a proposed window, then all the sensors in the group will fail consensus and no data will be returned at all (even if the other sensors in the group have data).

Synced: when true, the sensor in the group must be within “Align Tolerance” seconds of all the other synced sensors in order for the group to be at consensus.

Following options are referenced in the logical conditions outlined below.

### Object Definitions

In order to satisfy the real time requirements, the buffering requires components such as, but not limited to, a circular buffer, index results, and a sensor slice.

### Circular Buffer

In an example embodiment, a state of the circular buffer is tracked using the following:

Cursor: cursor refers to an index in the buffer marking the end of the data that has already been consumed.

Extent: extent refers to an index in the buffer marking the end of the new data that has been added (e.g., data that has not been consumed yet).

Capacity: capacity refers to a maximum number of samples that the buffer can hold.

Filled: filled refers to a total number of samples that are added to the buffer. This number can exceed capacity since the buffer gets reused over and over again.

### Index Results

In an example embodiment, each time a window search is performed for any sensor, multiple indices are tracked within the circular buffer. The multiple indices include:

Start: the time in seconds representing the start of the window for this sensor.

Stop: the time in seconds representing the end of the window for this sensor.

Min Stop: the time in seconds representing the earliest possible end of the window for this sensor. This satisfies the “Minimum Window” option, but not the “Window” option.

Stride: the time in seconds that becomes the “Start” time of the next window.

Start Index: the index in the circular buffer for this sensor that is closest to the “Start” time in seconds.

Stop Index: the index in the circular buffer for this sensor that is closest to the “Stop” time in seconds.

Min Stop Index: the index in the circular buffer for this sensor that is closest to the “Min Stop” time in seconds.

Stride Index: the index in the circular buffer for this sensor that is closest to the “Stride” time in seconds.

Have Window: true if able to find a window of data within the circular buffer that satisfies either the “Start” and “Stop” or the “Start” and “Min Stop” criteria.

N: the length of the found window in samples.

Split: true if the window wraps around the circular buffer so that it has data at the end of the buffer and at the beginning of the buffer.

New Cursor: on the next round of sensor merging, the location where the Cursor in the circular buffer is to be moved to.

Outcome: one of the values in the Outcomes section below.

Consensus: one of the values in the Consensus section below.

Group Start: if any of the sensors begins to lead or lag from the group, this is the start time the rest of the group agrees on. The leader or lagger sensor needs to try to match with this.

### Sensor Slice

A sensor slice indicates a single moment-in-time view of the circular buffer matching a certain start and stop time criteria as well as consensus information for the other sensors being streamed together. The sensor slice has the following definitions to keep track of its state.

Previous: Previous refers to one of the Index Results objects. Previous represents the results of the previous round of logical conditioning on the raw sensor samples.

Current: Current refers to one of the Index Results objects. Current represents the results of the current round of logical conditioning on the raw sensor samples.

Proposed: Proposed refers to one of the Index Results objects. Proposed represents the proposed round of logical conditioning on the raw sensor samples. The proposed condition will eventually become current after the consensus has been calculated.

Consumed: when true the data available in this slice was actually returned to whoever is using the combined sensor streams. If any of the consensus mechanisms fails, then the slice will not have been consumed, even if it was found and looked good. The logical conditioning finds a fit across all the sensors in the group. Some of them may have perfectly conforming data to the requirements, but if a required sensor is missing data, then they must all fail together.

Each time the logical conditioning is run, a new round of sensor slice objects is created to determine whether synchronized data is available to be returned.

### Outcomes

In an example embodiment, each round of logical conditioning for combining the multiple sensor streams starts with finding a subset of the circular buffer (for each sensor) that fits within the start and stop time stamps defining the slice of time of interest. Due to the various constraints described above, many things can go wrong. Accordingly, the possible outcomes for finding the subset of the circular buffer may be as follow:

Regular: the start, stop and stride index were found in the circular buffer.

Wrapped: the start, stop and stride index were found, but the stop index occurs before the start index in the circular buffer.

Failed: unable to find the stop index in the buffer, but a start index was found.

Short: the actual period of time found represented by the samples found within the target start and stop times is too short relative to the minimum window size option.

New Short: the number of seconds of new data found within the target start and stop times is too short for the minimum new window option.

No Stride: unable to find data at stride seconds after the current start time, which was supposed to be the start of the next round's window, but the data does not exist yet.

Stride Gap: would be the same as No Stride, except that a regular window was found, meaning that there is a large gap after the start time. However, since data exists before the stop time, and the stride exists between them, adjustment is made to start after the gap.

No Data: the circular buffer is empty or does not have any data.

No Start: could not find an index in the buffer corresponding to the start time, including the stop time. In other words, no start indicates it is impossible to find a window.

Min Regular: able to find start, stop and stride index in the circular buffer. However, the length in time of the data found only satisfies the minimum window size criteria, not the full window size.

Min Wrapped: same as for “Min Regular”, but the stop index occurs before the start index because it is wrapped around the circular buffer.

Few Samples: able to find start, stop and stride index in the circular buffer, but the number of samples within the time slice is too small for the minimum number of samples options.

### Stride Advancement

The decision on whether the window should stride forward for all sensors is complicated. It needs to take into account the consensus from the previous round of indexing and buffering as well as the outcomes from finding data within the previous set of conditions. Stride advancement decisions propose a new start and stop time for a window of data from each sensor. In an example embodiment, the following conditions are evaluated in an order below. If there is not an else section, just continue onto the next item in the list. For these logical conditions, the values of the “Proposed” result are set within the Sensor Slice for each sensor.

### Consensus

Consensus between multiple sensor streams results in each sensor having one of the following statuses:

Accept: all the required sensors have sufficient data within the window.

Reject: one of the required sensors has no data, consensus for all sensors is rejected.

Lag: this sensor has a data window, but its bounds in time lag the rest of the group.

Lead: this sensor has a data window, but its bounds in time lead the rest of the group.

Group Lag: one or more sensors is lagging the group.

Group Lead: one or more sensors are leading the group.

Group Fix: there is one or more sensors leading the group and one or more sensors lagging the group.

Unsynced: this sensor is not required to be time synced with the rest of the group in consensus.

### Frame Appending

In an example embodiment, the frame appending loop adds new sensor frames (or samples) to the circular buffer maintained for each sensor. Frames may arrive alone or in groups. The arrival of frames is independent of the time stamps for data contained within the frames. Thus, for handling gaps, the appending of frames is logically decoupled from the processing of the circular buffers of sensor samples. Once a set of frames is appended to the circular buffers for each sensor, all the necessary logical conditions described herein are computed to decide whether a valid window of data exists for all required sensors within the given start time and stop time.

After deciding whether a window of data can be returned, in an example embodiment, the state of the sensor samples within the circular buffers for all sensors is validated to decide whether or not enough remaining data exists in the circular buffers to warrant another round of logical condition checks for returning windows. This loop is repeated until the circular buffers' logical state has been exhausted and requires the appending of additional frames.

If the logical conditions associated with the various cases as described above are not addressed, then the logical conditions may cause an infinite loop and data is never returned again since it never returns to the frame appending step.

Accordingly, these conditions are checked to determine whether the circular buffers have sufficient data to run again. Further, these conditions are checked for every required sensor in the group, and all sensors in the group need to return True in order for the frame appending loop to continue without appending additional sensor frames. These conditions should be checked in the order they are presented. If there is no “else” clause, just step to the next numbered condition.

### New Data Computation

If a window is determined via consensus to be complete for all sensors, then the new indices in each window are computed as described herein.

Accordingly, in real time applications, where it is difficult to ensure that all the multiple sensor streams are aligned in time and with respect to the logical constraints required by applications or downstream users consuming the data, the disclosed embodiments provide the multiple logical conditions required to satisfy all of these corner cases with respect to real time, multimodal streaming of sensor values.

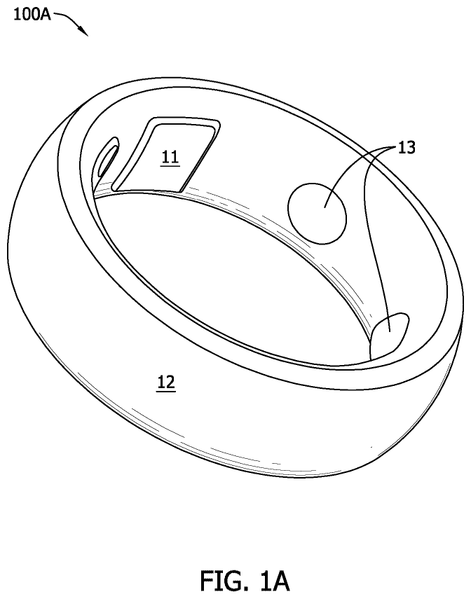

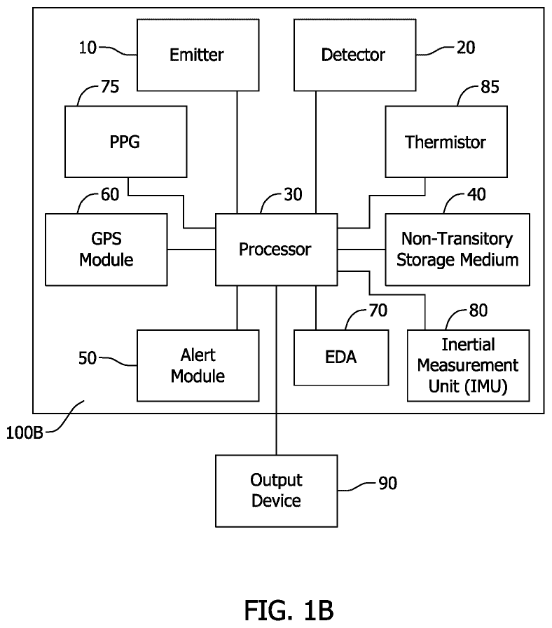

FIG. 1A illustrates an example wearable electronic device 100A. The wearable electronic device 100 may be worn by a user or secured to the user. By way of an example, the wearable electronic device may include, but is not limited to, a wearable computer, a wearable watch, a wearable communication device, a wearable media player, a wearable health monitoring device, and the like. In one example, the wearable electronic device 100A may be a wearable electronic device including multiple functionalities such as time keeping, health monitoring, sports monitoring, medical monitoring, stress monitoring, sleep monitoring, communications, navigation, computing operations, monitoring a user's physiological and/or biological signals and providing health-related information based on those signals, and/or communicating with other electronic devices or services in a wired or wireless manner. The wearable electronic device 100A may also provide alerts to the user, which may include one or more of audio, haptic, visual and/or other sensory output. The wearable electronic device 100A may also display data on a display device of the wearable electronic device 100A and acquire sensor data form one or more sensors positioned at or within the wearable electronic device 100A.

The wearable electronic device 100A includes a device body 11 including a housing that carries, encloses, and supports both externally and internally various components (including, for example, integrated circuit chips and other circuitry) to provide computing and functional operations for the wearable electronic device 100A. The components may be disposed on the outside of the housing, partially within the housing, through the housing, completely inside the housing, and the like. The housing may, for example, include a cavity for retaining components internally, holes or windows for providing access to internal components, and various features for attaching other components. The housing may also be configured to form a water-resistant or water-proof enclosure for the device body 11. For example, the housing may be formed from as a single unitary body and the openings in the unitary body may be configured to cooperate with other components to form a water-resistant or water-proof barrier. By way of a non-limiting example, the device body 11 may include components such as, but not limited to, processing units, memory, display, sensors, biosensors, speakers, microphones, haptic actuators, batteries, and so on. The wearable electronic device 100A may also include a band 12 or strap or other means for attaching to a user. In the example wearable electronic device 100A, the band 12 has an annular shape with an aperture to receive a finger of a subject user.

Additionally, the device body 11 may have one or more contact areas 13 for cognitive stress measurement for the user. By way of an example, the one or more contact areas may be provided as one or more buttons on the sides of the device body 11. Additionally, or alternatively, one or more EDA sensors, one or more temperature sensors, and/or one or more PPG sensors may be positioned on the bottom of the device body 11 such that these sensors come into contact with skin of the subject user or face the skin on the body for measuring various physiological and/or biological events.

FIG. 1B illustrates components of the wearable electronic device 100A according to an example of this disclosure. As shown in FIG. 1B, the wearable electronic device 100B includes an emitter 10 and detector 20, which are coupled to a processor 30. The processor 30 is coupled to a non-transitory storage medium 40. The wearable electronic device 100B is coupled to an output device 90. The wearable electronic device 100B is the wearable electronic device 100A shown in FIG. 1A.

The emitter 10 delivers light to a tissue and the detector 20 collects the optically attenuated signal that is backscattered from the tissue. In at least one example, the emitter 10 can be configured to emit at least three separate wavelengths of light. In another example, the emitter 10 may be configured to emit at least three separate bands or ranges of wavelengths. In at least one example, the emitter 10 may include one or more light emitting diodes (LEDs). The emitter 10 may also include a light filter. The emitter 10 may include a low-powered laser, LED, or a quasi-monochromatic light source, or any combination thereof. The emitter may emit light ranging from infrared to ultraviolet light. As indicated above, the present disclosure uses Near-Infrared Spectroscopy (NIRS) as a primary example and the other types of light can be implemented in other examples and the description as it relates to NIRS does not limit the present disclosure in any way to prevent the use of the other wavelengths of light.

The data generated by the detector 20 may be processed by the processor 30, such as a computer processor, according to instructions stored in the non-transitory storage medium 40 coupled to the processor. The processed data can be communicated to the output device 90 for storage or display to a user. The displayed processed data may be manipulated by the user using control buttons or touch screen controls on the output device 90 or on the device body 11.

The wearable electronic device 100B may include an alert module 50 configured to generate an alert. The processor 30 may send the alert to the output device 90 or the alert module 50 may send the alert directly to the output device 90. In at least one example, the wearable electronic device 100B may be configured so that the processor 30 is configured to send an alert to the output device 90 without the device including an alert module 50.

The alert may provide notice to a user, via a speaker or display on the output device 90, of a change in biological indicator conditions or other parameter being monitored by the wearable electronic device 100B, or the alert may be used to provide an updated biological indicator level to a user. In at least one example, the alert may be manifested as an auditory signal, a visual signal, a vibratory signal, or combinations thereof. In at least one example, an alert may be sent by the processor 30 when a predetermined biological indicator event occurs during a physical activity.

In at least one example, the wearable electronic device 100B may include a Global Positioning System (GPS) module 60 configured to determine geographic position and tagging the biological indicator data with location-specific information. The wearable electronic device 100B may also include an EDA sensor 70, a PPG sensor 75, an IMU 80, and a thermistor 85. The IMU 80 may be used to measure, for example, gait performance of a runner or pedal kinematics of a cyclist, as well as physiological parameters of a user during a physical activity. The EDA sensor 70, the PPG sensor 75, IMU 80, and thermistor 85 may also serve as independent sensors configured to independently measure parameters of physiological threshold. The wearable electronic device 100B may also include other types of sensors not described herein.

In one example, the application may be detection of a sleep onset which requires sensor data from thermistor 85, IMU 80, PPG sensor 75 and EDA sensor 70. Accordingly, thermistor 85, IMU 80, PPG sensor 75 and EDA sensor 70 may define a group of sensors, whose sensor data or sensor streams are synchronized as described herein.

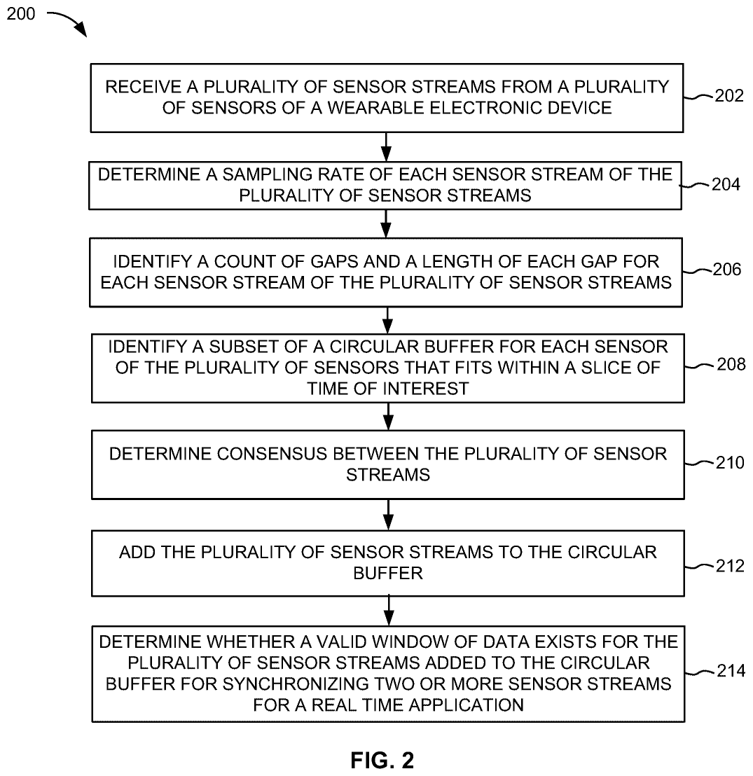

FIG. 2 is a flow-chart 200 of method operations for synchronizing two or more sensor streams of a plurality of sensor streams. Each sensor stream of the plurality of sensor streams corresponds with a different sensor of a plurality of sensors disposed at or within a body of the wearable electronic device 100A shown in FIG. 1A or 100B shown in FIG. 1B. The method operations include, receiving 202 the plurality of sensor streams. Each sensor stream of the plurality of sensor streams is received from a sensor of the plurality of sensors of a wearable electronic device. The method operations include determining 204 a sampling rate of each sensor stream of the plurality of sensor streams. By way of an example, the sampling rate is determined based upon a specification or sensor configuration of each sensor. Alternatively, or additionally, the sampling rate may be determined based on a total number of samples received in a specific time duration (e.g., one second). The method operations include identifying 206 a count of gaps and a length of each gap for each sensor stream. In the disclosure above, a gap and a length of gap in a sensor stream are discussed in detail, and, therefore, those details are not repeated again for brevity.

The method operations include identifying 208 a subset of a circular buffer for each sensor of the plurality of sensors. As sensor stream from each sensor is being fitted in a respective circular buffer of each sensor, a slice of time of interest and a corresponding subset of the circular buffer may be identified or determined. As described herein, the slice is characterized by a start timestamp and a stop timestamp. The method operations include determining 210 consensus between the plurality of sensor streams, as described herein, to proceed to adding 212 the plurality of sensor streams to the circular buffer of their respective sensor. The method operations include determining 214 whether a valid window of data exists the plurality of sensor streams added to the circular buffer for synchronizing two or more sensor streams of the plurality of sensor streams for a real time application, as described in detail above.

In an example embodiment, one or more sensor frames with raw data in one or more sensor streams of the plurality of sensor streams are arriving sporadically with contents whose timestamps may differ considerably from an arrival time of each the one or more sensor frames. A sensor frame with raw data in a first sensor stream of the plurality of sensor streams may have amount of data different or inconsistent from a sensor frame with raw data in a second sensor stream of the plurality of sensor streams. Additionally, or alternatively, a sensor frame in a first sensor stream of the plurality of sensor streams may include raw data before a gap in time, and wherein a sensor frame in a second sensor stream of the plurality of sensor streams includes raw data after a gap in time. The plurality of sensor streams may include at least one required sensor stream and at least one optional sensor stream.

As described herein, upon determining the valid window of data exists, the window of data or the data in the window is returned, and the window is slides forward in time by a stride value to cause an overlap between the returned data each time the valid window is returned. Further, the returned data may include data of at least one synchronized sensor stream and at least one unsynchronized sensor stream. Additionally, or alternatively, the returned data may be rejected upon determining the returned data is missing sensor data of required sensors. The returned data may allow for a “Minimum Window” size that grows until it reaches the full “Window” size. Further, the returned data may allow for a full “Window” size that shrinks when a gap is encountered until at most “Minimum Window” seconds of data are returned, and the returned data may allow for a “Minimum New Window” constraint where windows are only accepted if they have at least a certain number of seconds of new data. The returned data may include or recover from a lagging sensor or a leading sensor that differs significantly from the mean “Start” time of a group of two or more sensors of the plurality of sensors. As described herein, the lagging sensor and the leading sensor changes based on an alignment tolerance specific to the real time application. By way of an example, the window of data includes data that has been returned before and new data that has never been returned, and the returned data may be empty if the returned data does not include at least a “Minimum Number of Samples.”

As used herein, the phrase “at least one of” preceding a series of items, with the term “and” or “or” to separate any of the items, modifies the list as a whole, rather than each member of the list. The phrase “at least one of” does not require selection of at least one of each item listed; rather, the phrase allows a meaning that includes at a minimum one of any of the items, and/or at a minimum one of any combination of the items, and/or at a minimum one of each of the items. By way of example, the phrases “at least one of A, B, and C” or “at least one of A, B, or C” each refer to only A, only B, or only C; any combination of A, B, and C; and/or one or more of each of A. B, and C. Similarly, it may be appreciated that an order of elements presented for a conjunctive or disjunctive list provided herein should not be construed as limiting the disclosure to only that order provided.

One may appreciate that although many embodiments are disclosed herein, that the operations and steps presented with respect to methods and techniques described herein are meant as exemplary and accordingly are not exhaustive. One may further appreciate that alternate step order or fewer or additional operations may be required or desired for particular embodiments.

Although the disclosure herein is described in terms of various exemplary embodiments and implementations, it should be understood that the various features, aspects, and functionality described in one or more of the individual embodiments are not limited in their applicability to the particular embodiment with which they are described, but instead can be applied, alone or in various combinations, to one or more of some embodiments, whether or not such embodiments are described and whether or not such features are presented as being a part of a described embodiment. Thus, the breadth and scope of the present description should not be limited by any of the exemplary embodiments described herein but is instead defined by the claims herein presented.

What is claimed is:

1. A computer-implemented method comprising:
receiving a plurality of sensor streams, each sensor stream of the plurality of sensor streams is received from a sensor of a plurality of sensors of a wearable electronic device;
determining a sampling rate of each sensor stream of the plurality of sensor streams;
identifying a count of gaps and a length of each gap for each sensor stream of the plurality of sensor streams;
identifying a subset of a circular buffer for each sensor of the plurality of sensors that fits within a slice of time of interest, wherein the slice is characterized by a start timestamp and a stop timestamp;
determining consensus between the plurality of sensor streams;
adding the plurality of sensor streams to the circular buffer; and
determining whether a valid window of data exists for the plurality of sensor streams added to the circular buffer for synchronizing two or more sensor streams of the plurality of sensor streams for a real time application.

2. The computer-implemented method of claim 1, wherein one or more sensor frames with raw data in one or more sensor streams of the plurality of sensor streams are arriving sporadically with contents whose timestamps differ considerably from an arrival time of each the one or more sensor frames.

3. The computer-implemented method of claim 1, wherein a sensor frame with raw data in a first sensor stream of the plurality of sensor streams has amount of data different or inconsistent from a sensor frame with raw data in a second sensor stream of the plurality of sensor streams.

4. The computer-implemented method of claim 1, wherein a sensor frame in a first sensor stream of the plurality of sensor streams includes raw data before a gap in time, and wherein a sensor frame in a second sensor stream of the plurality of sensor streams includes raw data after a gap in time.

5. The computer-implemented method of claim 1, wherein the plurality of sensor streams includes at least one required sensor stream and at least one optional sensor stream.

6. The computer-implemented method of claim 1, further comprising returning the valid window of data, and sliding the window forward in time by a stride value to cause an overlap between the returned data each time the valid window is returned.

7. The computer-implemented method of claim 6, wherein the returned data includes data of at least one synchronized sensor stream and at least one unsynchronized sensor stream.

8. The computer-implemented method of claim 6, further comprising rejecting the returned data upon determining the returned data is missing sensor data of required sensors.

9. The computer-implemented method of claim 6, wherein the returned data allows for a “Minimum Window” size that grows until it reaches the full “Window” size.

10. The computer-implemented method of claim 9, wherein the returned data allows for a full “Window” size that shrinks when a gap is encountered until at most “Minimum Window” seconds of data are returned.

11. The computer-implemented method of claim 9, wherein the returned data allows for a “Minimum New Window” constraint where windows are only accepted if they have at least a certain number of seconds of new data.

12. The computer-implemented method of claim 9, wherein the returned data recovers from a lagging sensor or a leading sensor that differs significantly from the mean “Start” time of a group of two or more sensors of the plurality of sensors.

13. The computer-implemented method of claim 12, wherein the lagging sensor and the leading sensor changes based on an alignment tolerance specific to the real time application.

14. The computer-implemented method of claim 6, wherein the window of data includes data that has been returned before and new data that has never been returned.

15. The computer-implemented method of claim 6, wherein the returned data is empty if the returned data does not include at least a “Minimum Number of Samples”.

16. A wearable electronic device comprising:
a body;
a plurality of sensors disposed at or within the body, the plurality of sensors including one or more of a thermistor, an EDA sensor, a PPG sensor, an accelerometer, and/or an inertial measurement unit (IMU) sensor;
at least one memory storing instructions; and
at least one processor communicatively coupled with the at least one memory and configured to execute the instructions to perform operations comprising:
receiving a plurality of sensor streams, each sensor stream of the plurality of sensor streams is received from a sensor of the plurality sensors;
determining a sampling rate of each sensor stream of the plurality of sensor streams;
identifying a count of gaps and a length of each gap for each sensor stream of the plurality of sensor streams;
identifying a subset of a circular buffer for each sensor of the plurality of sensors that fits within a slice of time of interest, wherein the slice is characterized by a start timestamp and a stop timestamp;
determining consensus between the plurality of sensor streams;
adding the plurality of sensor streams to the circular buffer; and
determining whether a valid window of data exists for the plurality of sensor streams added to the circular buffer for synchronizing two or more sensor streams of the plurality of sensor streams for a real time application.

17. The wearable electronic device of claim 16, wherein one or more sensor frames with raw data in one or more sensor streams of the plurality of sensor streams are arriving sporadically with contents whose timestamps differ considerably from an arrival time of each the one or more sensor frames.

18. The wearable electronic device of claim 16, wherein a sensor frame with raw data in a first sensor stream of the plurality of sensor streams has amount of data different or inconsistent from a sensor frame with raw data in a second sensor stream of the plurality of sensor streams.

19. The wearable electronic device of claim 16, wherein a sensor frame in a first sensor stream of the plurality of sensor streams includes raw data before a gap in time, and wherein a sensor frame in a second sensor stream of the plurality of sensor streams includes raw data after a gap in time.

20. A wearable electronic device comprising:
an annular body;
a plurality of sensors disposed at or within the annular body, the plurality of sensors including one or more of a thermistor, an EDA sensor, a PPG sensor, an accelerometer, and/or an inertial measurement unit (IMU) sensor;
at least one memory storing instructions; and
at least one processor communicatively coupled with the at least one memory and configured to execute the instructions to perform operations comprising:
receiving a plurality of sensor streams, each sensor stream of the plurality of sensor streams is received from a sensor of the plurality sensors;
determining a sampling rate of each sensor stream of the plurality of sensor streams;
identifying a count of gaps and a length of each gap for each sensor stream of the plurality of sensor streams;
identifying a subset of a circular buffer for each sensor of the plurality of sensors that fits within a slice of time of interest, wherein the slice is characterized by a start timestamp and a stop timestamp;
determining consensus between the plurality of sensor streams;
adding the plurality of sensor streams to the circular buffer; and
determining whether a valid window of data exists for the plurality of sensor streams added to the circular buffer for synchronizing two or more sensor streams of the plurality of sensor streams for a real time application.
